(19) United States
(12) Patent Application Publication
(10) Pub. No.: US 2006/0019236 A1
(43) Pub. Date: Jan. 26, 2006

(54) Membrane fusion assay

(76) Inventors: Rory Rorison Duncan, Musselburgh (GB); Michael Alan Cousin, Edinburgh (GB)

Correspondence Address: MYERS BIGEL SIBLEY & SAJOVEC, PO BOX 37428, RALEIGH, NC, 27627, US

(21) Appl. No.: 11/170,513

(22) Filed: Jun. 29, 2005

(30) Foreign Application Priority Data
Jun. 30, 2004 (GB) GB0414653.6

(51) Int. Cl. C12Q 1/00; C12N 5/08

(52) U.S. Cl. 435/4; 435/368

(57) ABSTRACT

The present invention relates to an assay for detecting membrane fusion. More particularly the present invention relates to assays for detecting exocytosis and/or endocytosis wherein membrane fusion is detected by an increase in fluorescence of a lipophilic fluorescent dye due to fluorescence resonance energy transfer (FRET) occurring from one lipophilic fluorescent dye to another lipophilic fluorescent dye.

### RELATED APPLICATION

The present application claims priority under 35 U.S.C. § 119 to Great Britain Application Number GB0414653.6, filed Jun. 30, 2004, the disclosure of which is incorporated by reference herein in its entirety.

### FIELD OF THE INVENTION

The present invention relates to an assay for detecting membrane fusion. More particularly the present invention relates to assays for detecting exocytosis and/or endocytosis.

### BACKGROUND OF THE INVENTION

Styryl dyes are polar molecules that reversibly insert into lipid bilayer membranes. These dyes are widely used to study the exocytosis and/or endocytosis of neurotransmitter-containing synaptic vesicles from central neurones. Exocytosis is the fusion of a synaptic vesicle with the plasma membrane (resulting in neurotransmitter release) whereas endocytosis is a retrieval process to reform vesicles from the plasma membrane after exocytosis. Styryl dyes such as FM1-43 insert into, but do not pass through the plasma membrane of neurones, where they become fluorescent. On stimulation of exocytosis the dye is accumulated into newly formed vesicles since it is part of the plasma membrane that is internalised during endocytosis. This process can be visualised since styryl dyes are fluorescent when in membrane, but importantly not when in solution. Thus endocytosis can be monitored as an increase in fluorescence once non-internalised dye has been washed off the plasma membrane. Exocytosis can also be monitored using styryl dyes by stimulating neurones that already have vesicles preloaded with dye. On vesicle fusion the dye departitions from the vesicle membrane into solution and exocytosis is monitored as a decrease in fluorescence.

However, the assays described above have a number of disadvantages for high throughput screens (HTS). One major problem is a high background fluorescent signal. This is due to incomplete dye washing from the plasma membrane, which results in a greatly reduced signal-to-noise ratio. Some workers have shown that adding a different styryl dye, FM4-64, can reduce this background. FM4-64 was proposed to work by quenching any remaining plasma membrane FM1-43-dependent fluorescence and thus increasing the signal-to-noise ratio from the vesicle-inserted FM1-43 versus plasma membrane-quenched dye.

A second drawback of conventional styryl dye assays of exocytosis is that they lack sensitivity at the higher end of the assay since a decrease of signal (towards the noise and background) is measured—exactly where the signal is most interesting. This is compounded by rapid photobleaching of the dyes.

Moreover, normally membrane “recycling” further complicates styryl dye secretion assays. This is the biological effect where vesicle membranes that have just fused with the plasma membrane are quickly recovered by endocytosis. This means that absolute fluorescence values are a balance of exocytosis, where the fluorescent dyes are lost, and endocytosis, where they are re-internalised.

It is amongst the objects of the present invention to obviate and/or mitigate at least one of the aforementioned disadvantages.

### SUMMARY OF THE INVENTION

Generally speaking, certain embodiments of the present invention are based in part on the observations by the present inventors that FM1-43 dependent fluorescence is quenched by fluorescence resonance energy transfer (FRET) from FM1-43 to FM4-64 and that by using multi-photon excitation it is possible to efficiently excite FM1-43 (donor) but not FM4-64 (acceptor). This results in no acceptor (FM4-64) fluorescence in the absence of energy transfer from the donor (FM1-43) and amounts to a large increase in the signal-to-background noise ratio.

The present invention provides an assay for detecting membrane fusion, the assay comprising the steps of:

a) providing a first membrane comprising a first lipophilic fluorescent dye associated therewith;

b) irradiating said membrane so as to excite said first lipophilic fluorescent dye;

c) allowing a second membrane comprising a second lipophilic fluorescent dye associated therewith to come into contact with said first membrane; and

d) detecting any membrane fusion by an increase in fluorescence of said second lipophilic fluorescent dye due to fluorescence resonance energy transfer (FRET) occurring from said first lipophilic fluorescent dye to said second lipophilic fluorescent dye.

Said first membrane may typically be a cell membrane, such as the membrane of a eukoryotic cell. However, said first membrane may be any surface comprising lipid moieties capable of forming a membrane like structure. Suitable examples include liposomes which are essentially vesicle like structures formed of bi-layers of lipid molecules and viruses. Lipids generally comprise hydrophilic and hydrophobic regions and so suitable lipophilic fluorescent dyes may be hydrophobic and/or hydrophilic in nature. If said dye is both hydrophobic and hydrophilic in nature, the dye may be said to be amphipathic.

If the membrane is a cell membrane, the first dye may be partitioned in the cell membrane by first allowing the cell to take up the dye internally. The cells may then be stimulated by way of a chemical such as potassium chloride depolarisation or by electrical means e.g. field stimulation depolarisation to induce exocytosis. Vesicles within the cell with entrapped dye are stimulated to fuse with the cell membrane and the dye within the vesicles may become associated/partitioned within the cell membrane. A washing step or steps may thereafter be employed to wash off and/or away any dye that is not suitably associated or partitioned within the cell membrane.

Said first lipophilic fluorescent dye may be a styryl dye such as FM1-43, FM2-10 or FM1-84 with fluoresce green available from molecular probes. Other lipophilic dyes, for example the carbocyanine dyes DiI (DiIC₁₈(3)) or DiO (DiOC₁₈(3)) may also be employed as donor or acceptor molecules.

Said first lipophilic fluorescent dye is capable of being excited by electromagnetic radiation such that said dye is converted to its excited state. When in this excited state, said dye is capable of acting as an energy donor wherein energy may be passed to an acceptor dye, as will be further described hereinafter. The skilled man is aware of an appropriate wavelength of electromagnetic radiation with which to irradiate and excite said first lipophilic dye. This depends on the dye being chosen, but may typically be between 450-500 nm. It is to be understood that the wavelength required to excite the donor dye should be a shorter wavelength than that required to excite the acceptor dye.

Typically a laser may be used to irradiate the first lipophilic dye, or alternatively an electron beam generator timed to the appropriate wavelength may also be used.

Although, a styryl dye such as FM1-43 generally possesses a maximum absorption wavelength of about 488 nm (i.e. the optimum wavelength to excite the dye), the present inventors have found that it is possible to use an emerging fluorescence technique called multiphoton excitation which may be used to efficiently excite a donor dye molecule such as FM1-43, but does substantially not excite an acceptor dye molecule such as FM4-64 (see for example Williams et al., Curr. Opinion in Chem. Biol, 5, 603-608). In this manner electromagnetic radiation of much longer and less harmful wavelengths may be used to irradiate cells comprising the first lipophilic dye in order to excite the dye. Moreover, this results in no acceptor fluorescence in the absence of the energy transfer from the donor, something not possible using conventional excitation techniques and which results in a large increase in the signal-to-background noise ratio. Thus, for example, the present inventors have observed that a wavelength of about 800 nm may be used to excite FM1-43.

The second membrane comprising the second lipophilic fluorescent dye may fuse with the first membrane such that the first and second fluorescent dyes may be brought into close (e.g. <4 nm) proximity. This may occur by the processes known as exo or endo-cytosis.

Lipid vesicles comprising the second fluorescent dye associated/partitioned within a leaflet of the lipid bi-layer may be generated within a cell, in which case, membrane fusion is observed as a consequence of exocytosis, or generated outside a cell and brought into contact therewith, in order to observe membrane fusion. In order to observe exocytosis of a cell or cells it is generally necessary to first add the second dye to said cell or cells such that the cell partitions within the outer membrane of the cells. The cells may then be depolarised by chemical, pharmacological or electrical means in order to cause fusion of vesicles containing the second dye. The cells may then be washed in order to remove any remaining second dye from the cell surface. The first dye may then be added to the cell(s) and allowed to partition into the outer cell membrane, before the cells are irradiated so as to excite the first dye. In this manner only to the first dye in the outer membrane will fluoresce. Upon stimulation (e.g. by chemical, pharmacological or electrical means) the vesicles comprising the second dye exocytose such that the lipid vesicle and outer cell membrane fuse, thereby bringing the first and second dyes into close proximity and the same leaflet of the outer cell membrane.

Once the first excited donor dye is in sufficient proximity, as calculated by the present inventors to be substantially <4 nm, FRET may occur and energy is transferred to the second fluorescent dye such that the second dye is converted to an excited fluorescent state. By using first and second dyes that fluoresce at different wavelengths, it is possible to detect when FRET occurs and the second dye fluoresces.

Preferred second lipophilic fluorescent dyes are dyes that are excited to fluoresce at a longer wavelength than said first lipophilic dye and are understood to be acceptor dyes (i.e. dye which may accept an energy from said first excited fluorescent dye, when is sufficiently close proximity). Examples of preferred dyes are FM4-64 and FM5-95 available from Molecular Probes, Inc.

Typically, in order for fusion to occur, said two membranes may be encouraged/induced to fuse using a chemical such as potassium chloride at 40-50 nm, or chemical, pharmacological or electrical means.

Fluorescence resonance energy transfer (FRET) is a distance-dependent interaction between the electronic excited states of two dye molecules in which excitation energy is transferred from a donor molecule to an acceptor molecule without emission of a photon. The efficiency of FRET is dependent on the inverse sixth power of the intermolecular separation, making it useful over distances comparable with the dimensions of biological macromolecules. Thus, FRET is an important technique for investigating a variety of biological phenomena that produce changes in molecular proximity. For a general teaching of FRET the reader is directed to for example, Periasamy A, Day RN, “Visualizing protein interactions in living cells using digitized GFP imaging and FRET microscopy.” Methods Cell Biol. 1999; 58: 293-314.

The assays of the present invention may be used to identify molecules or agents (e.g. drug candidate) which may effect membrane fusion. The assay may also be adapted to a format such as a plate format known in the art, to allow many assays to be conducted simultaneously and to be conducted as a high-throughput screen. The assay may therefore be used to screen the activities of agents that potentially modulate neurotransmitter release (e.g. serotonin), amine release (e.g. histamine), G-protein-coupled receptor (GPCR) mediated secretion, or agents that affect ion channel activities. Moreover, the assay may be used to identify agents that act as agonists or antagonists of naturally occurring ligands. The assay may also be used to identify endocrine disruptors, as this is relevant in the environmental protection area, where it is thought that chemicals might mimic or block the effects of oestrogens, androgens and/or thyroid hormones in animals and/or humans. Further application may be in the field of developing anti-virals, where the disruption of virus membranes and cell membranes is studied.

In this manner, the assay generally may comprise the further step (typically between steps (b) and (c) of adding an agent to the assay system, so that when compared to a control assay (i.e. without added agent), the effect of the agent on membrane fusion can be observed.

Detection of fluorescence from said second lipophilic dye can be correlated with membrane fusion. In order to quantify the degree of membrane fusion, it is possible using known techniques, see for example “Optical analysis of synaptic vesicle recycling at the frog neuromuscular junction”, Bewick, G and Betz W, Science 10; 255 (5041):200-3, and as described hereinafter to determine total cell fluorescence over a time period, or more preferably the rate at which fluorescence occurs. For example, if a control assay identified a particular rate of fluorescence, an increase in the rate of fluorescence would be indicative of an enhancement of membrane fusion, whilst a decrease in the rate would indicate a reduction/inhibition of membrane fusion. This would be of use, for example, in identifying drugs which can increase or decrease neurotransmitter release.

In a further aspect there is provided a kit for use in an assay according to the present invention, the kit comprising:

eukaryotic cells capable of accepting a donor lipophilic fluorescent dye within the cell membrane of the cells;

a donor lipophilic fluorescent dye capable of being excited at a specific wavelength; and

an acceptor lipophilic fluorescent dye capable of accepting energy from said excited donor lipophilic fluorescent dye by way of fluorescence resonance energy transfer.

Typical eukaryotic cells include primary neurons such as cerebellar granule cells and other secretory cell lines such as hippocampal neurons PC12 cells and B104 cells.

The donor lipophilic fluorescent dye may be FM1-43, FM2-10 or FM1-84. Preferably the dye is FM1-43.

The acceptor lipophilic fluorescent dye may be FM4-64 or FM5-95. Preferably the dye is FM4-64.

FM1-43 and FM4-64 are members of a larger family of styryl dyes that differ in their hydrophobicity and spectral properties. FM1-43 homologues such as FM2-10 & FM1-84 can also act as donors to FM4-64. Similarly the FM4-64 homologue, FM5-95, can also act as an acceptor for the FM1-43 derivatives.

The hydrophobicity of styryl dyes determine the departition time in membrane, and without being bound by theory, it is predicted that this may also have implications for the FRET-based assay of the present invention. It is predicted that the best donors and acceptors would be the most hydrophobic dyes (FM1-84 & FM4-64) since they are partitioned in membrane for the maximal amount of time. This would allow the maximum possible time for FRET responses to occur upon membrane fusion before departitioning of the probes.

Typically the eukaryotic cells may be coated and/or adhered to a substrate such as the wall of a microtitre plate comprising 64, 128, 256 or 512 wells.

The present invention will now be described further by way of example and with reference to the Figures which show:

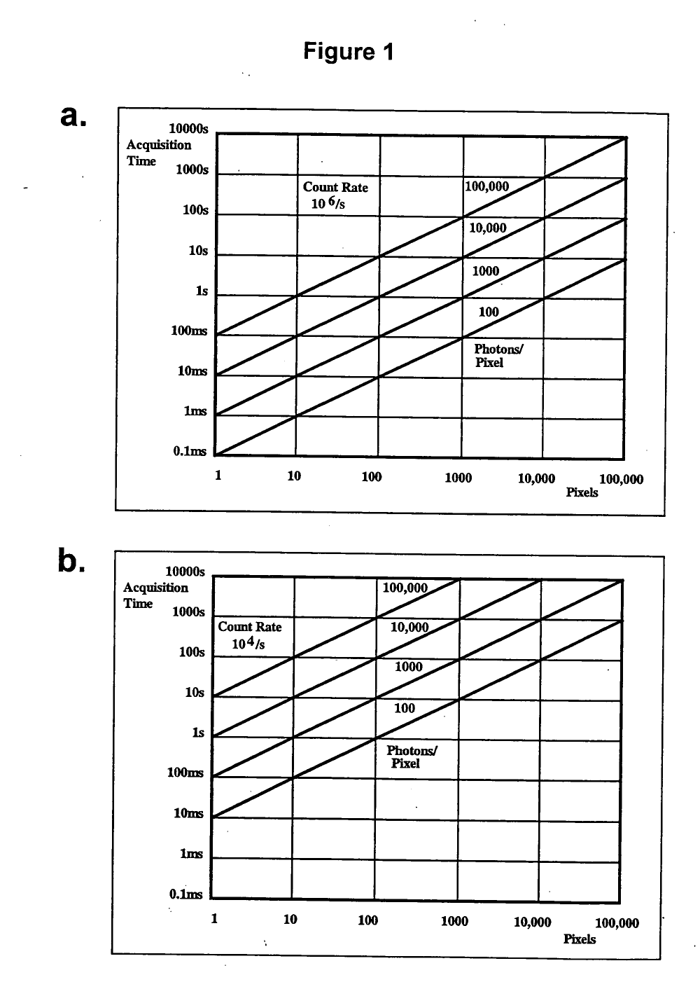

FIG. 1 shows a plot showing the acquisition time as a function of the number of pixels in the image. (a) The dependencies of scan time vs. pixel number for a count rate of 10⁶ S⁻¹, such count rates are typical for cellular autofluorescence or for samples of poor photostability. (b) For a count rate of 10⁴ S⁻¹; such count rates are typically for cellular autofluorescence or for samples of poor photostability.

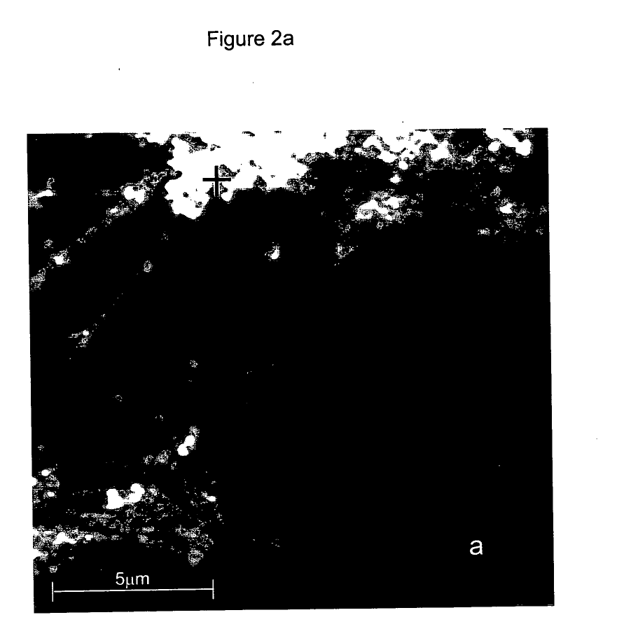

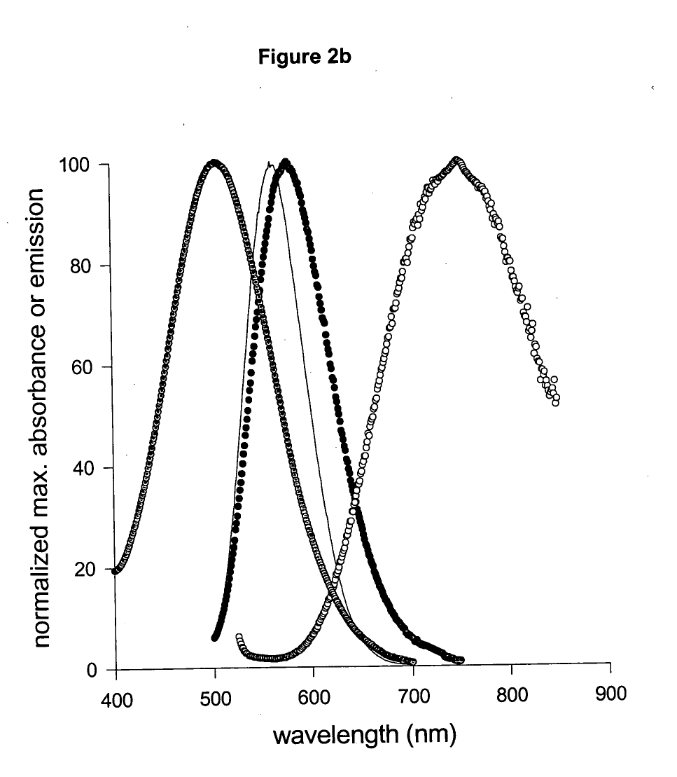

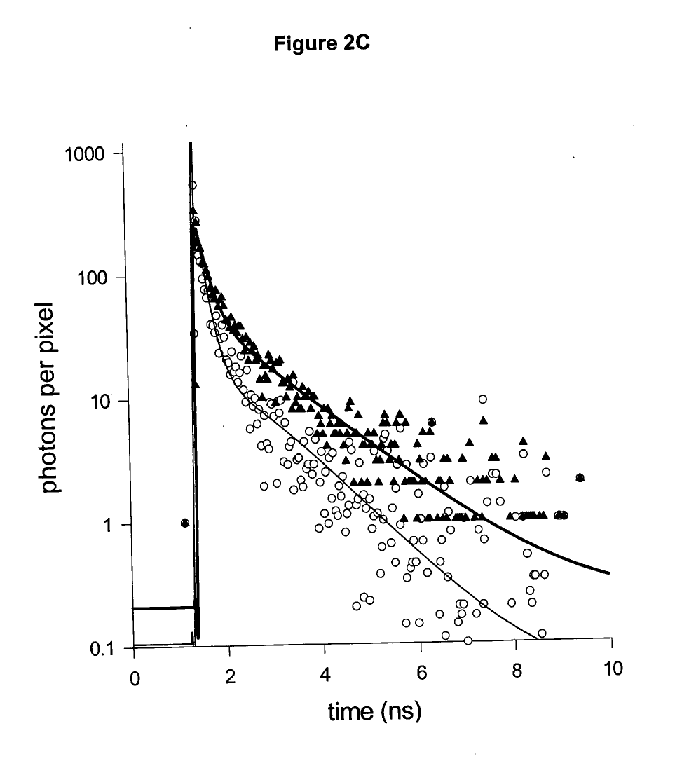

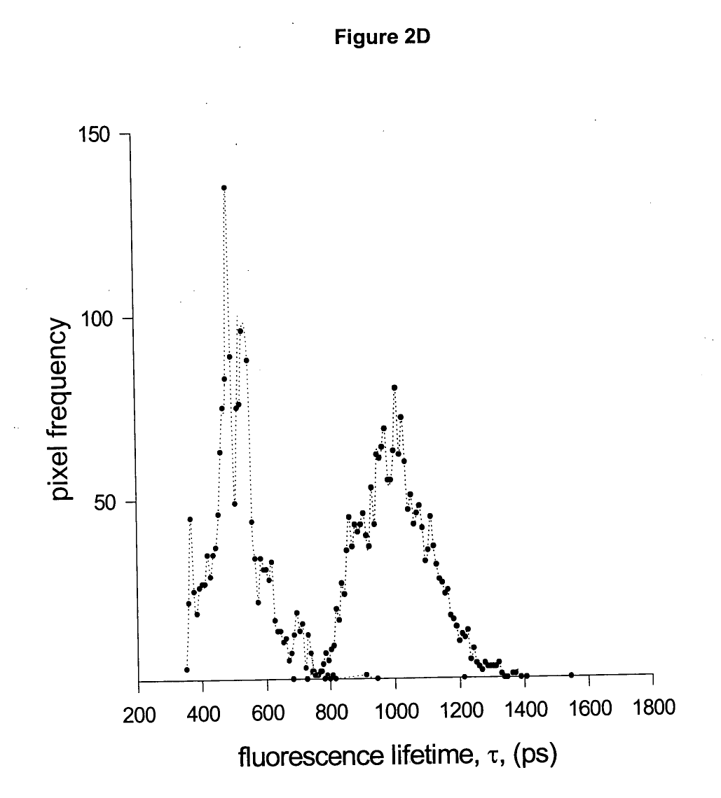

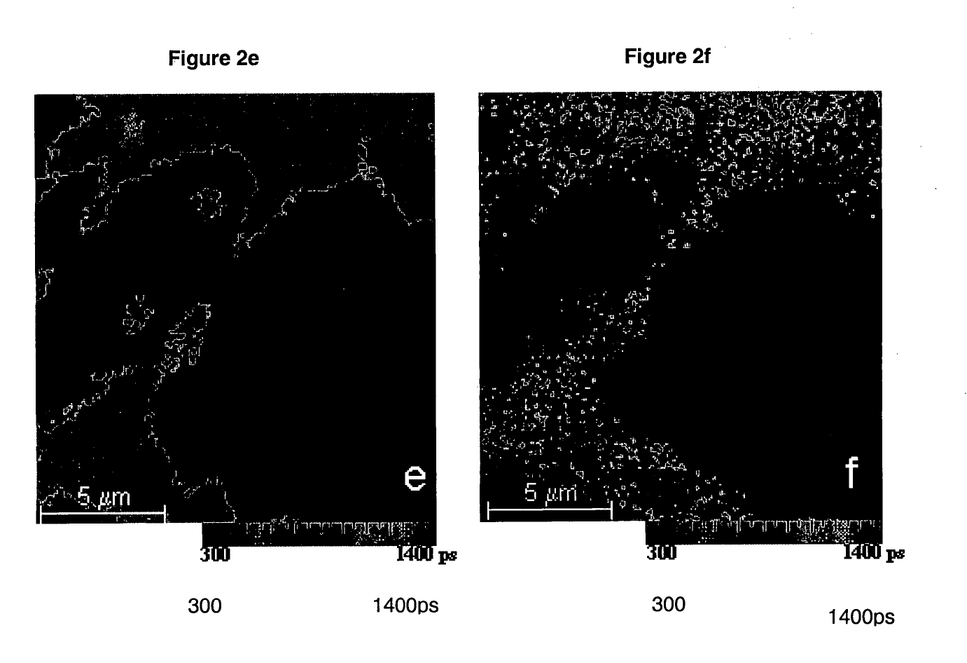

FIG. 2 shows living cerebellar cells (CGCs) were stained with FM1-43 and imaged using 800 nm TPE. The dye partitioned into membrane structures as expected (a). (b) The fluorescence spectra for membrane-intercalated FM1-43 and FM4-64, predicting that FM4-64 would act as an acceptor for FM1-43 energy; FM4-64 normalized absorbance, green filled circles, FM1-43 normalized emission, red filled circles, FM4-64 normalized emission, open circles. The line plot is the integrand, namely the product of the acceptor absorption and the donor emission, multiplied by the donor wavelength, λ⁴. (c) Decay curves from a 128×128 pixel fluorescence lifetime imagine microscopy (FLIM) image, using a 435-485 nm BP filter to dissect FM1-43 emission (filled circles, two-binned pixels underneath the cross in a), fit to a bi-exponential curve (black line). The same pixel was sampled after the same cells were counter stained with FM4-64 (open filled circles) and the normalized data fit to a bi-exponential decay (red line, six-binned pixels). (d) The resulting donor mean fluorescence lifetime, τ, frequency distributions before (black filled circles and line) and after (red filled circles and line) FM4-64 counter staining. (e,f) The FLIM maps generated from the distribution data for non-FRET and FRET image, respectively. In these FLIM maps, the image data brightness values have been equalized to reveal a donor-specific decrease in τ without a loss of image clarity due to intensity quenching. (g,h) Three-dimensional plots of photon counts against x, y position. 800 nm TPE was used as before, with a 500-550 bp emission filter to resolve FM1-43 (i.e. donor) fluorescence. Images were made using a Zeiss Plant NeoFLUAR 1.3 NA 40×oil-immersion objective lens; and

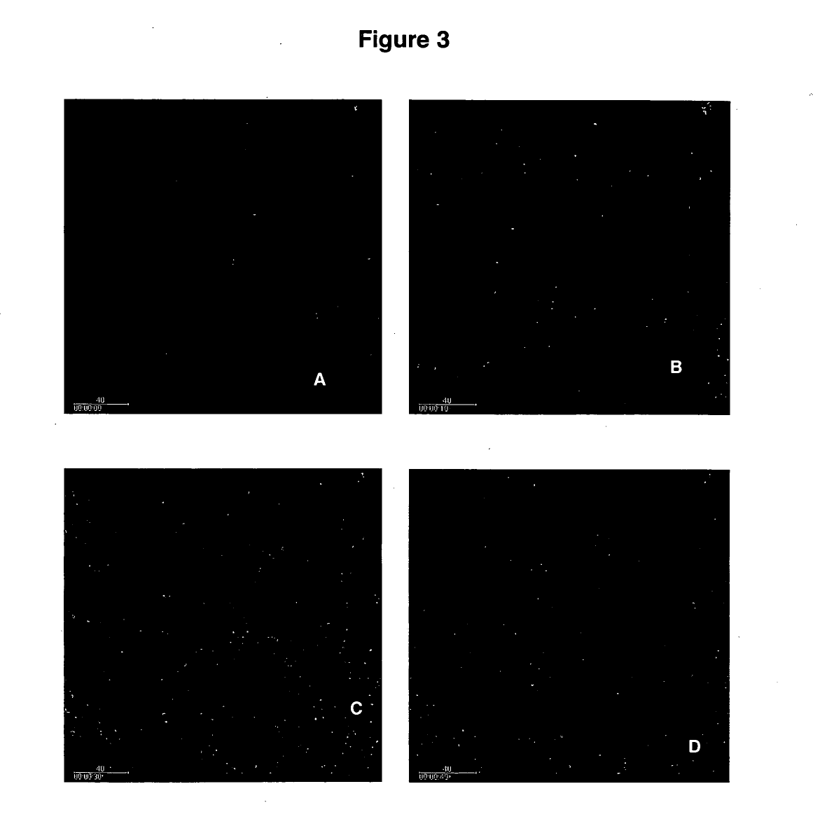

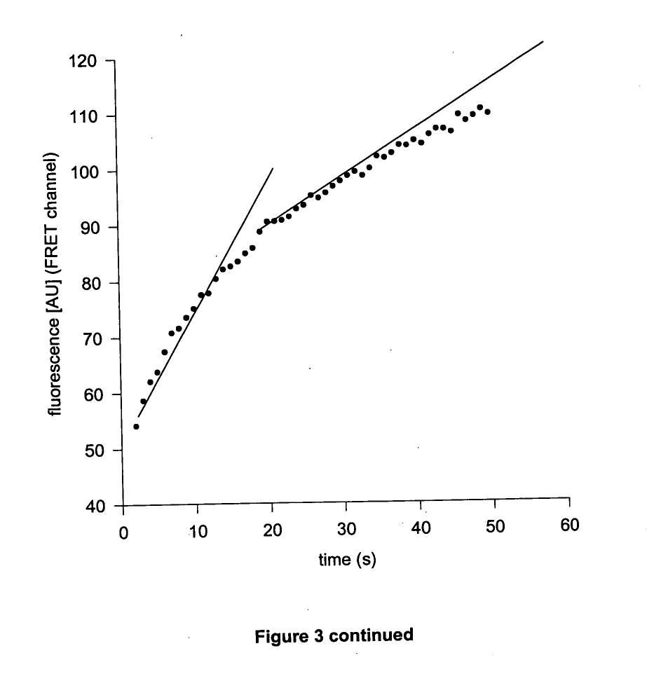

FIG. 3 shows the demonstration of FRET between FM1-43 (donor) and FM4-64 (acceptor) upon stimulation of exocytosis.

### MATERIALS & METHODS

Cerebellar granule cell cultures were prepared essentially as previously described (Courtney et al., 1990). Cells were plated on 42×0.17 mM poly-D-lysine coated glass coverslips at a density of 0.25×10⁶ cells/coverslip and cultured in minimal essential medium (MEM) containing Earle's salts (Gibco) plus 10% (v/v) fetal calf serum, 25 mM KCl, 30 mM glucose, 2 mM glutamine, 100 U mL⁻¹ penicillin and 100 μg mL⁻¹ streptomycin at 37° C., in a humidified atmosphere of 5% CO₂: 95% air. Culture medium was supplemented with 10 μM cytosine arabinoside after 24 h in vitro.

Styryl Dye Staining

Either FM1-43 (N-(3-triethylammoniumpropyl)-4-(4-(dibutylamino)styryl) pyridinium dibromide; Molecular Probes) or FM4-64 (N-(3-triethylammoniumpropyl)-4-(6-(4-(diethylamino) phenyl) hexatrienyl) pyridinium dibromide; Molecular Probes) were applied to neurones, either separately or sequentially, at 10 μM for 2 min to allow partitioning into the plasma membrane. Cells were washed twice in incubation medium (in mm: 170 NaCl, 3.5 KCl, 0.4 KH₂PO₄, 20 TES (N-tris[hydroxy-methyl]-methyl-2-aminoethanesulphonic acid), 5 NaHCO₃, 5 glucose, 1.2 Na₂SO₄, 1.2 MgCl₂, 1.3 CaCl₂, pH 7.4, 37° C.) to remove non-partitioned dye.

Fluorescence Imaging

All imaging experiments were performed using a Zeiss LSM 510 Axiovert confocal laser scanning microscope, equipped with a pulsed excitation source (MIRA 900 Ti:Sapphire femtosecond pulsed laser, with a coupled VERDI 10-W pump laser (Coherent, Ely, U.K.)). The laser was tuned to provide a TPE wavelength of 800 nm. Live cells on glass coverslips (37 mM) were imaged using an incubation chamber (H. Saur, Reutlingen, Germany). TPE data acquisition was performed using 512×512- or 1024×1024-pixel image sizes, with 4× frame averaging, using a Zeiss Plan NeoFLUAR 1.3 NA 40× oil-immersion, or a Zeiss C-Apochromat 1.2 NA 63× water-corrected immersion objective lens. Band pass (BP) and long pass (LP) emission filters were used, as detailed in the text, in conjunction with a Schott (New York, N.Y., U.S.A.) BG39 IR filter to attenuate the TPE light.

TCSPC-FLIM

TCSPC imaging requires that the scan control pulses of the microscope, i.e. the frame clock, line clock and, if possible, the pixel clock pulses, be available. All newer microscopes have access to these signals. Although the standard PMTs of the microscope can generally be used for TCSPC they do not yield an instrument response function (IRF) shorter than 500 ps full width half-maximum (Fwhm). It is therefore better to attach a fast detector at a suitable optical output of the microscope. TCSPC measurements were made under 800 nm TPE, using a non-descanned detector (Hamamatsu R3809U-50; Hamamatsu Photonics UK Ltd, Herts., U.K.) multi-channel late-photomultiplier tube (MCP-PMT), coupled directly to the rear port of the Axiovert microscope and protected from room light and other sources of overload using a Uniblitz hutter (Rochester, N.Y., U.S.A.). This MCP-PMT is a key to measuring very fast fluorescent lifetimes as it achieves a transit time spread (TTS; the limiting factor for TCSPC measurements) of 30 ps, and is free of afterpulses. Dark count rates were 10²-10³ photons per second. The MCP-PMT was operated at 3 kV, and signal pulses were pre-amplified using a Becker & Hickl HFAC-26 26-dB, 1.6-GHz preamplifier. TCSPC recording used the “reversed start stop” approach, with accurate laser synchronization using a Becker & Hickl SPC-730 card together with a PHD-400 reference photodiode, routinely at 79.4 MHz. In contrast to conventional TCSPC devices, the SPC boards use a novel analog-to-digital (AD) conversion (ADC) technique that cancels the unavoidable errors of an ultra-fast ADC chip. Together with a speed-optimized time-amplitude-converter (TAC), this achieves an overall dead time of only 125 ns per photon. BP and LP filters were used, as detailed in the text, to enable spectral separation of donor and acceptor FRET- and sensitized-emissions. Schott BG39 filters of 3-6 mm were positioned directly in front of the MCP-PMT. TCSPC recordings were acquired routinely for between 5 s and 25 s; mean photon counts were between 10⁵ and 10⁶ counts per second. Images were recorded routinely with 128×128 pixels, from a 512×512 scan, with 256 time bins per pixel, or 256×256 pixels from a 1024×1024 image scan with 64 time bins.

FLIM Data Analysis and FRET Calculations

Off-line FLIM data analysis used pixel-based fitting software (SPCImage, Becker & Hickl), able to import the binary data generated with the FLIM module.

The fluorescence was assumed to follow a multi-exponential decay. In addition, an adaptive offset correction was performed. A constant offset takes into consideration the time-independent baseline due to dark noise of the detector and the background caused by room light, calculated from the average number of photons per channel in front of the rising part of the fluorescence trace. To fit the parameters of the multi-exponential decay to the fluorescence decay trace measured by the system, a convolution with the instrumental response function was carried out. The optimization of the fit parameters was performed by using the Levenberg-Marquardt algorithm, minimizing the weighted chi-square quantity.

Steady-State Fluorescence Image Data Analysis

Steady-state image data deconvolution, manipulation and analyses used Huygens Pro Software (Scientific Volume Imaging, the Netherlands) running on Silicon Graphics Octane 2 workstations (SGI, CA, U.S.A.). Figure preparation used Imaris Srpass (Bitplane AG, Zurich) and Adobe Photoshop.

### RESULTS

Features of the TCSPC Imaging Technique

Time resolution. The time resolution of TCSPC techniques is given by the transit time spread in the detector. A system response shorter than 30 ps FWHM is achieved with MCP PMTs, which in conjunction with the minimum time channel width in the TCSPC modules of 813 fs, allowed lifetimes down to a few picoseconds to be determined (see below).

Acquisition time. TCSPC data were acquired at the full scanning rate of the microscope, with scan times of approximately 900 ms at 512×512 frame size. The FLIM image was recorded by accumulating over several frames with a lower pixel resolution of 128×128 pixels. Using a fibre-coupled detector proved to be unsatisfactory due to the poor efficiency of photon transmission; approximately 100-fold fewer photons were counted per pixel per second compared with the use of a direct, non-descanned couple to the rear port of the microscope. Non-descanned detection resulted in mean photon counts of ˜10⁴-10⁶ per second (Elangovan et al., 2002; van Kuppeveld et al., 2002). FIG. 1 shows the acquisition time as a function of the number of pixels in the image. FIG. 1(a) shows the dependencies of scan time vs. pixel number for a count rate of 10⁶ S⁻¹. Count rates of this order require highly fluorescent samples of good photostability. FIG. 1(b) is for a count rate of 10⁴ S⁻¹; such count rates are typical for cellular autofluorescence or for samples of poor photostability.

Acquisition times commonly used in live-cell frequency domain fluorescence lifetime measurements are about 5 s (Elangovan et al., 2002; van Kuppeveld et al., 2002). Although a single exponential lifetime analysis requires only 185 photons per pixel for an accuracy of 10%, double exponential analysis can require much higher photon numbers (Kollner & Wolfram, 1992). For a double-exponential decay analysis of our data we found about 1000 photons per pixel to be sufficient. Hence a count rate of 10⁶ S⁻¹ allowed the examination of about 5000 pixels with double-exponential decay analysis or approximately 50 000 pixels for single-exponential fitting (FIG. 1a). The count rates given above are averaged values for the whole image. However, commonly, a considerable fraction of the 128×128=16834 pixels remain relatively dark. This fact reduces the number of pixels that have to be taken into consideration for the analysis, thus increasing the effective number of photons within the analysed pixels. If the number of photons is low, an additional spatial binning can be performed; at an average count rate of 10⁴ S⁻¹ (FIG. 1b) a spatial binning of 3×3 increases the number of photons per pixel by almost an order of magnitude.

Because the total measurement time per pixel at this resolution did not exceed 20 μs, relatively fast acquisition times for a limited region of interest are feasible. An area of 50×50 pixels with a spatial binning of 3×3 pixels, for example, would require measurement times of only 50 ms, approaching typical video frame rates. Such measurements of restricted regions of interest within cells are commonly used to examine cellular dynamics in “real-time” (Steyer et al., 1997; Duncan et al., 2003).

Determination of Styryl Dye Fluorescence Lifetimes

FM1-43 and FM4-64 are amphiphilic probes that partition with lipid bilayers but do not cross them. These probes are highly fluorescent when partitioned in membranes but have little fluorescence in solution. The dyes are often used in combination in the study of membrane dynamics in secretory cells and neurones (Betz & Bewick, 1992; Cochilla et al., 1999; Cousin & Robinson, 1999; Straub et al., 2000; Zenisek et al., 2002), where FM4-64 may be used to quench undesirable FM1-43 fluorescence (Cochilla et al., 1999). Rat cerebellar granule cells (CGCs) are the most abundant neurone in the brain and can be cultured to a very high degree of homogeneity (>95%; Courtney et al., 1990). CGCs are glutamatergic and their axons and nerve terminals form the parallel fibres in the molecular layer of the cerebellum that synapse with Purkinje cells.

In order to determine the intercompartmental distance between these small molecular dyes in a plasma membrane, CGCs were stained using FM1-43 and imaged using 800 nm TPE. The steady-state fluorescence image (FIG. 2a) demonstrated that FM1-43 dye localized in membrane structures in these cells as expected. TCSPC data were acquired using a 20 s recording time and BP and LP filters, revealing a biexponential decay for FM1-43 (a₁=48±5%, τ₁=0.26±0.1 ns; a₂=50±3%, τ₂=1.79±0.14 ns), with a mean lifetime of 0.98±0.13 ns (χ²=1.15; n=3; FIG. 2b-d; Table 1).

TABLE 1
TCSPC data for FM1-43 and FM1-43/FM4-64 co-labelling.
The symbols are as for Table 1. The data were acquired
from the same FM1-43n cells before and after counter-labelling
with FM4-64 (see Methods).

| Group | α1 (%) | τ₁ (ps) | α₂ (%) | τ₂ (ps) | τ(ps) | n |
|---|---|---|---|---|---|---|
| FM1-43 | 48 ± 5 | 261 ± 106 | 50 ± 4 | 1796 ± 135 | 989 ± 133 | 3 |
| FM1-43/ | 73 ± 2 | 163 ± 64 | 26 ± 5 | 1291 ± 234 | 496 ± 72 | 3 |
| 4-64 | | | | | | |

λ_{excitation} = 800 nm TPE,
λₑₘᵢₛₛᵢₒₙ = 500-550 nm BP.

Subsequent staining of the cells using FM4-64, which has previously been postulated to be an efficient acceptor for FM1-43 resonance energy (Rouze & Schwartz, 1998), quenched the steady-state fluorescence emission of the latter (FIG. 2b,c,e). Furthermore, using 800 nm TPE, we were able to resolve completely the FM1-43 emission from FM4-64, as the latter did not absorb excitation energy under these conditions. Thus, no FM4-64 emission was detected using this wavelength. When both dyes were present in the same intracellular compartment, i.e. the outer leaflet of the plasma membrane, strong FM4-64 emission was detected. To support the conclusion that this was sensitized emission due to FRET from FM1-43, we performed TCSPC as before using a 435-485 nm BP or a 500-550 nm BP filter to resolve donor lifetime data. These data were compared with those acquired from the same cells prior to FM4-64 staining, confirming that the FM1-43 emission was quenched, and that the mean donor lifetime was reduced from 0.98±0.1 ns to 0.49±0.07 ns (t-test; P<0.0001, n=3, FIG. 2c-e). These data were used to calculate an apparent FRET efficiency value of 0.5. The FM1-43/FM4-64 co-staining (donor only) data were best fit to a bi-exponential decay (FIG. 2c), with a quenched-donor (FM1-43) fast lifetime component of 0.16±0.06 ns. The long lifetime component of quenched FM1-43 was 1.29±0.23 ns (FIG. 2c-f). This effect was only resolved using this filter arrangement (800 nm TPE, 500-550 bp emission) to dissect donor lifetime from sensitized acceptor emission.

The donor lifetime data combined with donor and acceptor spectra were used to calculate the distance between the two fluorophores (Förster, 1948; Stryer & Haugland, 1967; Stryer, 1978; Patterson et al., 2000b). The Förster distance (R₀) for donor energy transfer from FM1-43 to FM4-64 was calculated to be 3.14±0.06 nm, with the standard error estimated to be 2% (by the propagation of errors method (Bevington, 1969)). The intermolecular distance between plasma membrane partitioned FM1-43 and FM4-64 was calculated using the mean lifetime components, resulting in a distance approximation, r, of 3.39 nm. The average FRET distance r was found to be 3.52 nm. However, an additional caveat should be noted here, in that the apparent donor quenching may be affected by the relative concentrations of the fluorophores. Nevertheless, the distance calculations are useful in reflecting the requirement for the two dyes to be partitioned in the same leaflet of the same membrane compartment for FRET to occur.

These data were supported by comparing the mean number of photons counted in each time bin for each TCSPC pixel before and after FM4-64 addition. This analysis revealed that the donor emission was reduced from a mean of ˜500 photons per pixel (no binning) in the FM1-43 samples to a mean of ˜200 photons per pixel in the FM4-64 quenched sample, representing a decrease in photon emission of ˜60%, in agreement with the reduction in τ.

FRET is exquisitely distance-sensitive, so that it can only occur between lipophilic fluorescent dyes such as FM1-43 and FM4-64 if they are in the same leaflet of the membrane i.e. the same membrane compartment (calculated by the present inventors as about <4 nm apart). The present FRET-based assay monitors the fusion of for example FM4-64-loaded vesicles with plasma membrane pre-saturated with FM1-43. On stimulation of vesicle fusion, energy will be transferred from donor (FM1-43) to acceptor (FM4-64) as both membranes merge. A FRET response under these conditions will be indicative of (1) secretion (e.g. of serotonin), and (2) voltage-gated ion channel activity. Since neuronal secretion requires the activation of voltage-sensitive ion channels, and this assay may also act as an indirect measurement of this cellular activity. Changes in the rate of exocytosis will be revealed as changes in the rate or the extent of the development of FRET (see FIG. 3), allowing the elucidation of agents which modulate exocytotic kinetics in cells.

The present FRET-based assay eliminates the problem associated with assays of the prior art relate to a decrease in signal, since the assay monitors the appearance of FRET-dependent FM4-64 emission on fusion of the synaptic vesicle. FIG. 3 shows FM4-64 emission (sensitized emission from energy donated by membrane-bound FM1-43) appearing in real time in nerve terminals after the cells have been stimulated to secrete. Thus FM4-64-loaded vesicles (not fluorescing) fuse with a plasma membrane containing fluorescent FM1-43. Thus instead of the usual decreasing signal, our approach quantifies the increase of sensitized emission from background to a high value as secretion proceeds. The rate of increase in acceptor fluorescence, demonstrating FRET and thus membrane fusion, describes the rate of exocytosis. Any modulation in exocytosis, for instance, by exogenous agents, would be reflected as a change in this rate (FIG. 1).

By using FRET, the fluorescence measured is only of membrane compartment mixing, and thus re-internalisation of both dyes simultaneously (as may be predicted) will have no effect on the readout. This may provide another advantage, as simultaneous analyses of donor and acceptor fluorescence may reveal selective FM1-43 uptake into vesicles that precedes FRET emission, thus providing a concurrent readout of both exocytosis and endocytosis.

The present inventors have therefore (1) shown that FRET mediates FM4-64-dependent quenching of FM1-43 fluorescence emission, (2) calculated that FRET can only occur if the dyes are in the same membrane compartment, and (3) identified specific wavelengths that abolish background fluorescence from non-FRET emission and used this information to develop novel membrane fusion assays.

A major drive for drug discovery programs is the analysis of agents that act as agonists or antagonists of naturally occurring ligands. Thus miniaturization of this FRET-based whole cell assay to a plate format can allow it to be employed as a high-throughput functional screen. The screen may be used to quantify the activities of agents that potentially modulate neurotransmitter release (e.g. serotonin), G-protein-coupled receptor (GPCR)-mediated secretion or agents that affect ion channel activities. Since these dyes are not membrane-selective, assays of the present invention can also be applied to any model system of membrane fusion, for example in vitro or in vivo assays of viral entry into cells (which is dependent on fusion of the viral envelope with the host plasma membrane).

A number of assays exist for quantifying secretion (exocytosis) or membrane fusion. However all HTS assays to some extent or another do not directly report the process of membrane fusion itself. However, the disclosed FRET-based assay is homogenous, simple and inexpensive. It will directly quantify membrane fusion and in addition provide readout of the intra-compartment distance between membranes.

There is an existing assay that monitors membrane voltage potential (to quantify voltage gated ion channel activity; VIPR from Aurora) but it is accepted to lack the capability to monitor ion channel kinetics in real time. Because of this, pharmacological intervention is employed to maintain channels in the “open” state. Ideally, a high-throughput screen should not compromise physiological relevance. Hence, perhaps a more appropriate method would activate voltage-gated ion channels by delivering physiologically relevant action potentials, via electrical stimulation, while simultaneously recording the downstream consequences of channel activity in a cell population. Once such event is exocytosis, where the present FRET-based assay will report changes in membrane fusion with high sensitivity.

### REFERENCES

Betz, W. J., & Bewick, G. S. (1992) Optical analysis of synaptic vesicle recycling at the frog neuromuscular junction. Science, 255, 200-203.

Bevington, P. R. (1969) Data Reduction and Error Analysis for the Physical Sciences. McGraw-Hill, New York.

Cochilla, A. J., Angleson, J. K. & Betz, W. J. (1999) Monitoring secretory membrane with FM1-43 fluorescence. Annu. Rev. Neurosci. 22, 1-10.

Courtney et al., (1990) The interactions between plasma membrane depolarization and glutamate receptor activation in the regulation of cytoplasmic free calcium in cultured cerebellar granule cells. J Neurosci. December 1990; 10 (12): 3873-9.

Cousin, M. A., & Robinson, P. J. (1999) Mechanisms of synaptic vesicle recycling illuminated by fluorescent dyes. J. Neurochem. 73, 2227-2239.

Duncan, R. R., Greaves, J., Wiegand, U. K., Matskevitch, J., Bodammer, G., Apps, D. K., Shipston, M. J & Chow, R. H., (2003) Functional and spatial segregation of secretory vesicle pools according to vesicle age. Nature, 422, 176-180.

Elangovan, M., Day, R. N. & Periasamy, A. (2002) Nanosecond fluorescence resonance energy transfer-fluorescence lifetime imaging microscopy to localize the protein interactions in a single living cell. J. Microsc. 205, 3-14.

Forster, V. T. (1948) (German). Ann. Phys. 6, 54-75.

Kollner, M. & Wilfram, J. (1992) How may photons are necessary for fluorescence-lifetime measurements?. Chem. Phys. Lett. 200, 199-204.

Patterson, G. H., Piston, D. W. & Barisas, B. G. (2000) Forster distances between green fluorescent protein pairs. Anal. Biochem. 284, 438-440.

Periasamy A, Day RN, “Visualizing protein interactions in living cells using digitized GFP imaging and FRET microscopy.” Methods Cell Biol. 1999; 58: 293-314.

Rouze, N. C. & Schwartz, E. A. (1998) Continuous and transient vesicle cycling at a ribbon synapse. J. Neurosci. 18, 8614-8624.

Steyer, J. A., Horstmann, H. & Almers, W. (1997) Transport, docking and exocytosis of single secretory granules in live chromaffin cell. Nature, 388, 474-478.

Straub, M., Lodemann, P., Holroyd, P., Jahn, R. & Hell, S. W. (2000) Live cell imaging by multifocal multophoton microscopy. Eur. J. Cell Biol. 79, 726-734.

Stryer, L. (1978) Fluorescence energy transfer as a spectroscopic ruler. Annu. Rev. Biochem. 47, 819-846.

Stryer, L. & Haugland, R. P. (1967) Energy transfer: a spectroscopic ruler. Proc. Natl. Acad. Sci. USA, 58, 719-726.

Williams et al., Curr. Opinion in Chem. Biol, 5, 603-608.

van Kuppeveld, F. J. W. J., Melchers, P. H., Willems & Gadella, Jr (2002) Homomultimerization of the coxsackievirus 2B protein in living cells visualized by fluorescence resonance energy transfer microscopy. J. Virol. 76, 9446-9456.

Zenisek, D., Steyer, J., Feldman, M. & Almers, W. (2002) A membrane marker leaves synaptic vesicles in milliseconds after exocytosis in retinal bipolar cells. Neuron, 35, 1085.

1. An assay for detecting membrane fusion, the assay comprising the steps of:
a) providing a first membrane comprising a first lipophilic fluorescent dye associated therewith;
b) irradiating said membrane so as to excite said first lipophilic fluorescent dye;
c) allowing a second membrane comprising a second lipophilic fluorescent dye associated therewith to come into contact with said first membrane; and
d) detecting any membrane fusion by an increase in fluorescence of said second lipophilic fluorescent dye due to fluorescence resonance energy transfer (FRET) occurring from said first lipophilic fluorescent dye to said second lipophilic fluorescent dye.

2. The assay of claim 1, wherein the first lipophilic dye is capable of being excited by multiphoton excitation and the second lipophilic dye is not capable of being excited by multiphoton excitation.

3. The assay of claim 2, wherein the first lipophilic dye is excited by electromagnetic irradiation at a wavelength substantially longer than the optimal excitation wavelength.

4. The assay of claim 1, wherein said first membrane is a cell membrane.

5. The assay of claim 4, wherein the first dye is partitioned in the cell membrane.

6. The assay of claim 5, wherein the cell is stimulated by a chemical such as potassium chloride or by electrical means.

7. The assay of claim 6 wherein the cells are washed to remove any dye that is not suitably associated or partitioned within the cell membrane.

8. The assay of claim 1, wherein said first membrane is a surface comprising lipid moieties capable of forming a membrane like structure.

9. The assay of claim 1, wherein said first membrane is a liposome.

10. The assay of claim 1, wherein said first lipophilic fluorescent dye is a styryl dye.

11. The assay of claim 1, wherein the first lipophilic fluorescent dye is selected from the group consisting of:
(i) FM1-43;
(ii) FM2-10; and
(iii) FM1-84

12. The assay of claim 11, wherein the first lipophilic fluorescent dye is FM1-43.

13. The assay of claim 1, wherein the first fluorescent dye is a carbocyanine dye.

14. The assay of claim 1, wherein the first fluorescent dye is either DiI (DiIC18(3)) or DiO (DiOC₁₈(3)).

15. The assay of claim 1, wherein the donor dye is excited by a shorter wavelength than that required to excite the acceptor dye.

16. The assay of claim 1, wherein the second lipophilic fluorescent dyes fluoresce at a longer wavelength than said first lipophilic dye.

17. The assay of claim 1, wherein the second lipophilic dye is FM4-64 or FM5-95.

18. The assay of claim 1, wherein the second lipophilic dye is FM4-64.

19. The assay of claim 1, wherein a laser is used to irradiate the first lipophilic dye.

20. The assay of claim 1, wherein an electron beam generator timed to the appropriate wavelength is used to irradiate the first lipophilic dye.

21. The assay of claim 1, wherein the first lipophilic dye is excited by electromagnetic radiation of a wavelength between 450-500 nm.

22. The assay of claim 1, wherein the second membrane comprising the second lipophilic fluorescent dye fuses with the first membrane via exo or endo-cytosis.

23. An assay for use in studying exocytosis, comprising the steps of:
(a) providing a cell comprising a second lipophilic fluorescent dye;
(b) inducing the formation of vesicles in said cell;
(c) adding a first lipophilic fluorescent dye;
(d) irradiating said cell so as to excite said first lipophilic fluorescent dye;
(e) stimulating the cell to induce exocytosis; and
(f) detecting exocytosis by an increase in fluorescence of said second lipophilic fluorescent dye due to fluorescence resonance energy transfer (FRET) occurring from said first lipophilic fluorescent dye to said second lipophilic fluorescent dye.

24. The assay of claim 23, wherein the first lipophilic dye is excited by multiphoton excitation and the second lipophilic dye is not capable of being excited by multiphoton excitation.

25. The assay of claim 23, wherein the first lipophilic dye is excited by electromagnetic irradiation at a wavelength substantially longer than the optimal excitation wavelength.

26. The assay of claim 23, wherein the cells are depolarised by chemical, pharmacological or electrical means.

27. The assay of claim 23, wherein the cells are washed in order to remove any remaining second dye from the cell surface.

28. The assay according to claim 1 for use in identifying molecules or agents which may effect membrane fusion comprising the step of, adding between steps (b) and (c) an agent to the assay and observing an effect the agent has on membrane fusion.

29. The assay of claim 28, wherein the effect on membrane fusion is compared to the membrane fusion occurring in a control assay (i.e. without added agent).

30. The assay of claim 28, wherein the first lipophilic dye is excited by multiphoton excitation and the second lipophilic dye is not capable of being excited by multiphoton excitation.

31. The assay of claim 28, wherein the first lipophilic dye is excited by electromagnetic irradiation at a wavelength substantially longer than the optimal excitation wavelength.

32. The assay of claim 28, wherein the second lipophilic fluorescent dyes fluoresce at a longer wavelength than said first lipophilic dye.

33. The assay of claim 28, wherein the donor lipophilic fluorescent dye is FM1-43, FM2-10 or FM1-84.

34. The assay of claim 28, wherein the donor lipophilic fluorescent dye is FM1-43.

35. The assay of claim 28, wherein the acceptor lipophilic fluorescent dye is FM4-64 or FM5-95.

36. The assay of claim 28, wherein the acceptor lipophilic fluorescent dye is FM4-64.

37. A kit for use in an assay according to the present invention, the kit comprising:
a donor lipophilic fluorescent dye capable of being excited at a specific wavelength; and
an acceptor lipophilic fluorescent dye capable of accepting energy from said excited donor lipophilic fluorescent dye by way of fluorescence resonance energy transfer when the dyes are sufficiently close; and
cells or membranes capable of accepting a donor lipophilic fluorescent dye.

38. The kit of claim 44, wherein the cells are eukaryotic cells

39. The kit of claim 44, wherein the eukaryotic cells are primary neurons.

40. The kit of claim 44, wherein the eukaryotic cells are secretory cell lines.

41. The kit of claim 44, wherein the eukaryotic cells are selected from the group consisting of:
(i) cerebellar granule cells;
(ii) hippocampal neurons;
(iii) PC12 cells; and
(iv) B104 cells.

42. The kit of claim 44, wherein the donor lipophilic fluorescent dye is FM1-43, FM2-10 or FM1-84.

43. The kit of claim 44, wherein the donor lipophilic fluorescent dye is FM1-43.

44. The kit of claim 44, wherein the acceptor lipophilic fluorescent dye is FM4-64 or FM5-95.

45. The kit of claim 44, wherein the acceptor lipophilic fluorescent dye is FM4-64.

46. The kit of claim 44, wherein the eukaryotic cells are coated and/or adhered to a substrate.

47. The kit of claim 44, wherein the eukaryotic cells are coated and/or adhered to a microtitre plate.
